(19) United States
(12) Patent Application Publication
(10) Pub. No.: US 2009/0187068 A1
(43) Pub. Date: Jul. 23, 2009

(54) DEVICE FOR SAMPLE TAKING OR TRANSFER IN GENITAL ORGANS WITH AN IMPROVED ULTRASOUND VISIBILITY

(75) Inventors: Patrick BOUVERET, Neuilly-En-Thelle (FR); Vincent BERJAT, Boran-Sur-Oise (FR)

Correspondence Address: YOUNG & THOMPSON, 209 Madison Street, Suite 500, ALEXANDRIA, VA, 22314, US

(73) Assignee: PRODIMED SAS, Neuilly-En-Thelle (FR)

(21) Appl. No.: 12/356,706

(22) Filed: Jan. 21, 2009

(30) Foreign Application Priority Data
Jan. 21, 2008 (FR) 08/00299

(51) Int. Cl. A61B 17/435

(52) U.S. Cl. 600/34

(57) ABSTRACT

A device whose main part is a catheter or probe which is advantageously made up by an echogenic tube on its whole length, and which is proportionally transparent. The structure is made up by a material chosen from among the group comprising amorphous thermoplastic polymer matrices incorporating a braid, strand or strip with a non metal echogenic material which is biocompatible and chosen from among filled or un-filled semi-crystalline thermoplastic polymers.

This invention relates to an echogenic medical device enabling one to take samples in the genital organs, such as samplings of body fluids or fragments of the inner walls of genital organs, or allowing the transfer of products, notably gametes or embryos, in female genital organs, notably in the uterus or uterine tubes (oviducts).

The sample taking devices which are now commonly used generally include a cylindrical tube made or one or several polymers, open at both ends, having an outside diameter between 1-3 millimetres and an inner diametre between 0.5-2.8 millimetres, for a length of 15-30 centimetres.

This cylindrical tube is shaped in such a manner:
that it may be linked through a connecting part (such as a Lüer or Lüer Lock cone) to a syringe, or to any other device allowing one to create a under- or overpressure, to collect or inject a product or a tissue.

The devices for the transfer of gametes or embryos which are currently used are intra-uterine probes generally comprising a transparent and flexible probe made of one or several polymers, having a length between 15-20 cm, and ending with a highly flexible catheter with a blunt head and having two opposing lateral openings or one single distal opening; a standard connecting part (such as a Lüer or Lüer Lock cone) provides for the connection of this device onto a syringe.

This kind of device allows one to carry out an intra uterine artificial insemination with previously prepared sperm. The probe makes for easier access to the uterine cavity without any injury, and makes it possible to liberate spermatozoids near tubal ostia.

As a variant, some embryo transfer devices include:
an introduction catheter, made of one or several polymers, with a length of ca. 15-25 cm, an outer diameter of ca. 2-3 mm, and which is advantageously equipped with a sliding ring (limit stop),
a polymer reimplantation catheter with a length of ca. 15-30 cm, with reference graduations separated from one another by one centimetre in its lower part.

Generally, disposable use of this known transfer device is as follows: the introducing catheter is pushed up to the inner opening of the cervix.

Simultaneously the embryo(s) has(have) been filled by the biologist into the reimplantation catheter.

This reimplantation catheter is then introduced into the introducing device, and will be pushed up to the transfer area.

When the first specific notch of the reimplantation catheter comes flush with the proximal end of the introducing device's Lüer cone, their two distal ends coincide. The notches as seen on the introducing device when looking from its distal end allow one to calculate the length which is introduced into the uterus. One must add the overhang of the reimplantation catheter (the number of cm by which the tally notches have been exceeded).

The devices for sample taking or transfer such as hereabove defined are difficult to locate in the uterine cavity, and the precision with which the embryos or gametes are left is a precondition for a high success rate of transfers—the same being true for the precision of sample takings—and several solutions have been suggested to improve the echogenicity of these devices, viz. their ability to be identified with ultrasounds.

Thus Application GB 2 379 610 describes the introduction of gas bubbles in the whole depth of the plastic material which is used in medical devices. International Application WO 00/09178 describes a medical device a part of which is made echogenic by the incorporation of metal microparticles with a size below 500 nm, and Application EP 1 462 056 discloses a catheter whose sheath comprises an outer layer containing isopentane gas microspheres, and an inner layer without bubbles. International Application WO 2005/035043 describes a shape memory device used as a vaso-occlusive device whose main part is a tube comprising a structure which is made up by a mainly magnetic and moderately echogenic material. This part may be made up by a thermoplastic polymer matrix, filled with metal particles (tungsten, palladium, gold, silver, iron oxide, etc..), or within which bubbles are trapped. The area which is thus obtained is opaque.

However the echogenicity of these various devics is not optimal, and there is a need to have devices with better performances.

The aim of the invention is to yield sample taking or transfer devices whose improved characteristic, in relation to hereabove-described existing devices, is to possess a more marked echogenic character on it whole length, and also near the useful opening of these devices for sample taking and transfer. The main part which makes up the catheter is worked out from a transparent tube which is made up of a thermoplastic polymer matrix, impregnated or formulated with a material which is able to generate acoustic discontinuities. This improvement allows one to manipulate the device easier and more finely during the medical procedure (with a positioning which is much more precise than that allowed by the above-described devices, in the area which is to be the site of sample taking or transfer). Moreover this allows the physician no longer to work in the blind, as is now the case. A better visualization allows one to position embryos with a high precision in the uterine cavity, thus helping improve the transfer success rate.

The aim of the invention is also to yield sample taking or transfer devices which are such, as compared with the above-described existing devices, that the echogenic material never comes into contact with the products which are taken or transferred, in order not to alter their biochemical characteristics nor their physiological condition.

The invention relates to a device whose main part is a catheter which is elaborated from an echogenic tube on its whole length, and which is proportionally transparent, said echogenic material being chosen within the group comprising amorphous thermoplastic polymer matrices within which a braid, a strand or a strip of non metal and biocompatible echogenic material is incorporated.

The term proportionally transparent means that the tube is not completely opaque, but that transparent areas are also present, the acoustic discontinuity being in relation to the different densities (compactness) of the two materials (impedance difference), viz. the polymer matrix and the material which makes up the braid, the strand or the strip. The thus obtained transparent areas allow a biologist to check, for instance, for the presence of embryos in the catheter before transfer, and also to assess the absence of these embryos after reimplantation, thus testifying to the success of the transfer.

Among amorphous polymer matrices one may notably cite polyether based aliphatic polyurethanes. These matrices are advantageously used when the device is to be used in a medical procedure.

The impregnated braid, strand or strip is made up by a non metal, biocompatible material, which is advantageously chosen from among semi-crystalline thermoplastic polymers (polyamides, polyesters for instance) or from among filled thermoplastic materials (barium sulfate or a borosilicate based compound for instance).

In a particular mode of the invention the braid is made up by a contiguous intertwining of strands in order to obtain a regular and adaptable geometry (meshing). Mesh density is controlled and adjustable if needed (variable width) in order to make it possible to visualize through the tube. The thus obtained tube is echogenic on its whole length and opaque because of the presence of a mesh and of regular and transparent area.

The impregnated braid is generally made up of a monofilament or strand whose diameter is advantageously between 0.05-0.10 mm.

Moreover the inner surface of the tube which makes up the catheter has a perfectly smooth topography, and this prevents any adhesion onto the inner surface. For this reason the devices as defined by the invention are particularly appropriate for the transfer of gametes or embryos, or for the taking of wall fragments. The quality of the echographic imagery is significantly improved, considering the increased echoes, and the different nature of acoustic discontinuities which are generated.

The inventive devices make it possible to take samples in the genital organs for analysis, such as body fluid samples, or fragments of the inner wall of male or female genital organs, more particularly female genital organs, notably near the cervix, the uterus or the oviducts, or to allow the transfer of products such as chosen from among gametes or embryos, or of substances or products which are used in radiology in the female genital organs, notably in the above-mentioned female genital organs.

The inventive devices are made up of a single-way catheter (single channel) which is open in its distal part near the useful opening, within which the fluid and the embryo circulate.

According to the invention a useful opening means the hole(s) through which, in the above-mentioned devices, samples which are taken are aspirated, or gametes, embryos, active substances or contrast substances are expelled.

In an advantageous embodiment of the invention the device includes:
the catheter corresponding to a cylindrical tube, whose distal end is:
   either open, thus making a useful opening for body fluid, cell fragment, tissue or genital organ wall sample taking, or for the transfer of products such as gametes, embryos, active substances or contrast products,
   or sealed, apart from at least a so-called sucking hole, which may be situated in a lateral position at the distal end of said catheter, thus making a useful opening for taking body fluid, cell fragment, tissue or genital organ wall samples, or for the transfer of products such as gametes, embryos, active substances or contrast products,
a water-tight plunger which is able to move in said cylindrical tube, and which is connected to the distal end of a rod having a proximal end advantageously equipped with a prehension organ, or of a syringe or another vacuum system making it possible to create an under-pressure or a over-pressure in the echogenic tube.

The length of the catheters for the above-mentioned sample taking, transfer or assays is preferably between ca. 150-ca. 300 mm.

Another aim of the invention is also the use of a device such as hereabove described in order to make it possible to take samples in the genital organs for analysis or to allow the transfer of products, notably embryos or gametes.

The inventive devices may be made according to any art technique. Thus the manufacturing process may include the following steps:
manufacturing of a braid if a braid is used,
manufacturing of echogenic tubes through extrusion and impregnation in the case of structures with a braid, or by co-extrusion in the case of structures with an echogenic strip of strand,
trauma-less extraction of fibers at the end of the thus obtained tubes.

In an advantageous embodiment of the inventive process, manufacturing the braid and impregnation in the tube are simultaneously undertaken in a single step through a thermoplastic polymer extrusion process. One may also, if advisable, generate microcavities during this step.

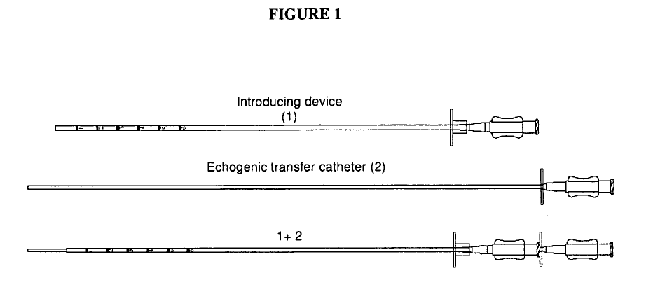

The invention will be described in more detail with the help of FIGS. 1-3.

FIG. 1 shows an example of a device for the inventive transfer of embryos. The introducing device (1) bears notches for non echogenic positioning.

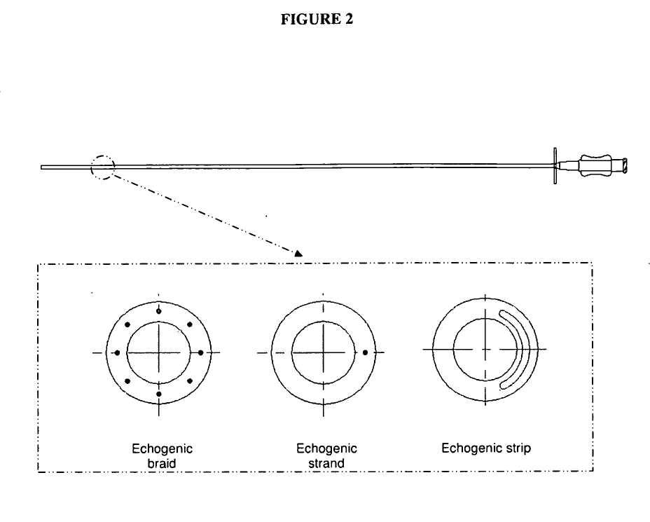

FIG. 2 shows a cross section of the echogenic transfer catheter through the presence of an echogenic braid, a strip or a strand.

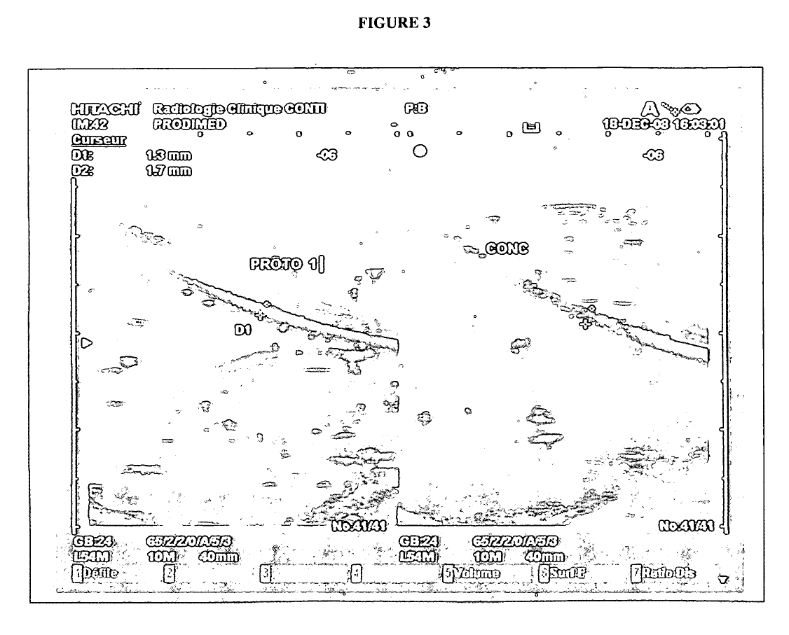

FIG. 3 is a comparative echography between the inventive device (PROTO 1) and a former art device (CONC).

The inventive devices possess a more marked echogenic character on their whole length, they are much more homogeneous, notably near the useful opening for sample taking or transfer.

1. A device, notably for the transfer of embryos, whose main part is a catheter which is manufactured from an echogenic tube on its whole length and which is proportionally transparent, said echogenic material being chosen from among the group of materials comprising amorphous thermoplastic polymer matrices, wherein a braid, a strand or a strip made of a metal and biocompatible echogenic material is incorporated.

2. An echogenic device according to claim 1, characterized in that the braid, the strand or the strip is made up by a non metal material chosen in the group comprising semi-crystalline thermoplastic polymers and filled thermoplastic materials.

3. A device according to claim 2, characterized in that the semi-crystalline thermoplastic polymer is chosen from among polyamides and polyesters

4. A device according to claim 2, characterized in that the filled thermoplastic materials are filled with particles which are selected from among barium sulfate and borosilicate based compounds.

5. An echogenic device according to claim 1, characterized in that the transfer or sample taking catheter is a tube which is made up by a polyether based aliphatic polyurethane amorphous thermoplastic polymer within which a non metal echogenic braid, strand or strip is incorporated, said device being specifically appropriate for medical uses.

6. An echogenic device according to claim 1, characterized in that the impregnated braid is made up of a single filament or strand whose diameter is between 0.01-0.10 mm, advantageously between 0.05-0.10 mm.

7. A device according to claim 1, characterized in that it includes:
the catheter which is a cylindrical tube whose distal end is:
either open, thus making a useful opening for taking body fluids, cell fragments, tissues or genital organ walls samples, or for the transfer of products such as gametes, embryos, active substances or radiological products,
or sealed, except at least one so-called aspiration hole, which may be situated in a lateral position of the distal end of said catheter, and which makes up the useful opening for taking body fluid, cell wall, tissue or genital organ wall samples, or for the transfer of products such as gametes, embryos, active substances or contrast products,
a water-tight plunger which is able to move within said cylindrical tube, and which is connected to the distal end of a rod whose proximal end isa davantageously equipped with a prehension organ, or of a syringe or other vacuum system allowing one to create an under-pressure or a over-pressure in the echogenic tube.
